(19) United States
(12) Patent Application Publication
(10) Pub. No.: US 2009/0097613 A1
(43) Pub. Date: Apr. 16, 2009

(54) NUCLEAR MEDICAL DIAGNOSIS DEVICE

(75) Inventor: Hiromichi Tonami, Kyoto (JP)

Correspondence Address: J C PATENTS, INC., 4 VENTURE, SUITE 250, IRVINE, CA, 92618, US

(73) Assignee: Shimadzu Corporation, Kyoto (JP)

(21) Appl. No.: 12/297,432

(22) PCT Filed: May 22, 2006
(86) PCT No.: PCT/JP2006/310136

§ 371 Date: Oct. 16, 2008

(51) Int. Cl. A61B 6/00

(52) U.S. Cl. 378/19

(57) ABSTRACT

A nuclear medical diagnostic device, for determining parameters T1, T2, and K required by a scintillator array identification mechanism without using a jig, includes a scintillator block formed by optically combining a plurality of self-radioactive scintillator arrays, in which the scintillator arrays have several scintillators that are two-dimensionally and compactly arranged and have respectively different attenuation time of a luminescence pulse in a γ-ray incident depth direction; a light receiving element for converting the luminescence pulse emitted from the scintillator block into an electric signal; an A/D converter for converting the electric signal outputted from the light receiving element to a digital signal; and an identification mechanism for identifying the scintillator arrays having respectively different attenuation time of the luminescence pulse in the γ-ray incident depth direction. The parameters required by the identification mechanism are determined according to a signal count of self-radioactivity of the self-radioactive scintillator arrays.

### BACKGROUND OF THE INVENTION

1. Field of the Invention

The present invention generally relates to a nuclear medical diagnostic device (an emission computed tomography (ECT) device), which applies an radioactive agent to a subject and concurrently measures a pair of γ-rays discharged by positron radioactive isotopes (RIs) accumulated in a target portion of the subject, so as to obtain a tomogram of the target portion. In particular, the present invention relates to a technique of counting γ-rays at the same time.

2. Description of Related Art

A positron emission tomography (PET) device is taken as an example to illustrate a nuclear medical diagnostic device, that is, an ECT device. The PET device is formed in the following manner. Opposite γ-ray detectors are used to detect two γ-rays at an angle of approximately 180° discharged from a target portion of a subject. When the γ-rays are detected (counted) at the same time, a tomogram of the subject is formed again. Further, some of the γ-ray detectors used to concurrently count the γ-rays in the PET device are formed by scintillators and photomultipliers. The scintillators will emit light after the γ-ray discharged from the subject is incident thereon, and the photomultipliers convert the light emitted by the scintillators to an electric signal.

In principle, the γ-rays discharged from a visual field centre are obliquely incident on the scintillators of the γ-ray detector D, as shown in FIG. 16. When the divided scintillators are not in the γ-ray incident direction, the γ-ray is detected at both the correct positions and the incorrect positions. That is, the visual difference error increases from the visual field centre to the peripheral parts, such that the tomogram obtained by the PET device becomes inaccurate. Therefore, as shown in FIG. 17, the scintillators are divided (optically combined) into scintillators having different attenuation time of the luminescence pulse in the γ-ray incident direction. For example, when a γ-ray detector MD is used, in which the scintillators are divided into a scintillator array having a shorter γ-ray attenuation time on a γ-ray incident side and a scintillator array having a longer γ-ray attenuation time on a photomultiplier side. Even if the γ-rays are obliquely incident on the scintillators of the γ-ray detector MD, the positions of the discharged γ-rays can still be detected accurately. Hence, a more accurate tomogram is obtained, and improvement is achieved (for example, refer to Japanese Patent Publication No. H06-337289 (Page 2 to 3 and FIG. 1), and Japanese Patent Publication No. 2000-56023 (Page 2 to 3 and FIG. 1)).

Further, the specific means for detecting the γ-ray position, having the scintillator array with the shorter attenuation time and the scintillator array with the longer attenuation time stacked in the γ-ray incident direction, includes the following mechanisms: an adding mechanism for converting an electric signal output from a light receiving element, that is, an analog signal SF (the signal of the scintillator array with the shorter attenuation time) or SR (the signal of the scintillator array with the longer attenuation time) into a digital signal by using an A/D converter as shown in FIG. 18, and adding sequentially the digital signals converted by the A/D converter as shown in FIG. 19; an identifying value calculating mechanism for calculating an identifying value representing a value A_{T1}/A_{T2} or B_{T1}/B_{T2} obtained by dividing an intermediate added value A_{T1} or B_{T1} by a total added value A_{T2} or B_{T2}, in which the intermediate added value A_{T1} or B_{T1} is a value obtained by adding the digital signals until the middle of a period starting from the luminescence pulse emitted from the scintillator block to the end of luminescence; in other words, until an intermediate moment in the adding mechanism. The total added value A_{T2} or B_{T2} is a value obtained, in the adding mechanism, by adding the digital signals during a period starting from the luminescence of the luminescence pulse emitted from the scintillator block to the end of luminescence; a mechanism for deciding a middle value K according to a maximum value and a minimum value in the identifying values calculated by the identifying value calculating mechanism; and an determination mechanism for determining whether an identifying value calculated by the identifying value calculating mechanism is greater or smaller than the middle value K.

Patent document 1: Japanese Patent Publication No. H06-337289 (Page 2 to 3 and FIG. 1)

Patent document 2: Japanese Patent Publication No. 2000-56023 (Page 2 to 3 and FIG. 1)

However, the existing nuclear medical diagnostic device has the following problems. That is, in the case of a two-stage scintillator detector 112, for example, having scintillator arrays of a two-stage structure as shown in FIG. 20, parameters T1, T2, and K required by the scintillator array identification mechanism are decided in the following manner. The two-stage scintillator detector 112 disposed in a dark box 115 starts to irradiate γ-ray on a front surface 110 of the scintillator array, and calculates a signal count N1 and a signal count N2 through a determination calculation, in which the signal count N1 is determined as the count of signals from the front surface 110 of the scintillator array, and the signal count N2 is determined as the count of signals from a back surface 111 of the scintillator array. Then, as shown in FIG. 21, the γ-ray is only irradiated on the back surface 111 of the scintillator array, and a signal count N2′ and a signal count N1′ are calculated through an determination calculation, in which the signal count N2′ is determined as the count of signals from the back surface 111 of the scintillator array, and the signal count N1′ is determined as the count of signals from the front surface 110 of the scintillator array. Further, as shown in FIG. 22, when a ray source is not used, and under a background of natural radioactive ray 116, a signal count N1b and a signal count N2b are calculated through a determination calculation, in which the signal count N1b is determined as the count of signals from the front surface 110 of the scintillator array, and the signal count N2b is determined as the count of signals from the back surface 111 of the scintillator array. Further, (N1-N1b)/(N2-N2b) and (N2′-N2b)/(N1′-N1b) are defined. When (N1-N1b)/(N2-N2b) and (N2′-N2b)/(N1′-N1b) are equal and become the maximum, this parameter is defined as the optimal parameter. In this case, a lead collimator jig 113 and an RI ray source 114 are required to only irradiate the γ-ray on any one scintillator array. Not only the operation is time-consuming, the management of the RI ray source 114 is difficult. Further, after the detector is installed in the PET device, a large lead collimator jig and RI ray source matching the PET device are required, and the operation is complicated.

### SUMMARY OF THE INVENTION

Accordingly, the present invention is directed to a nuclear medical diagnostic device, which is capable of easily determining the parameters required by a scintillator array identification mechanism by using self-radioactive scintillator arrays.

As broadly embodied and described above, the nuclear medical diagnostic device of the present invention includes a scintillator block formed by optically combining a plurality of self-radioactive scintillator arrays, in which the plurality of scintillator arrays has a plurality of scintillators that is two-dimensionally and compactly arranged, and has different attenuation time of a luminescence pulse in a γ-ray incident depth direction; a light receiving element for converting the luminescence pulse emitted from the scintillator block into an electric signal; an A/D converter for converting the electric signal output from the light receiving element, that is, an analog signal, into a digital signal; and an identification mechanism for identifying the plurality of scintillator arrays having different attenuation time of the luminescence pulse in the γ-ray incident depth direction. The parameters required by the scintillator array identification mechanism are decided with reference to a signal count of the self-radioactivity of the self-radioactive scintillator arrays.

### [Effect of the Invention]

The nuclear medical diagnostic device of the present invention includes a scintillator block formed by optically combining a plurality of self-radioactive scintillator arrays, in which the plurality of scintillator arrays has several scintillators that are two-dimensionally and compactly arranged, and has different attenuation time of a luminescence pulse in a γ-ray incident depth direction; a light receiving element for converting the luminescence pulse emitted from the scintillator block into an electric signal; an A/D converter for converting the electric signal output from the light receiving element, that is, an analog signal, into a digital signal; and an identification mechanism for identifying the plurality of scintillator arrays having different attenuation time of the luminescence pulse in the γ-ray incident depth direction. The parameters required by the scintillator array identification mechanism are decided with reference to a signal count of the self-radioactivity of the self-radioactive scintillator arrays. In the case that such an identification mechanism is used, the radioactivity of the self-radioactive scintillators is fixed; therefore, once the data of NF and NR are obtained, other lots of the scintillators can also use the same data of NF and NR. That is, once the data of NF and NR are obtained, the lead collimator jig and RI ray source used only for irradiating γ-rays on any one scintillator array as in the previous examples are not required. Hence, the operation is simplified, and the management of the RI ray source becomes unnecessary. Further, after a detector is practically loaded on the PET device, the large lead collimator jig and RI ray source matching the PET device are not required, so the operation is simple.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is an outside view of a radiation detector of the present invention.

FIG. 2 shows a scintillator array identification mechanism of the radiation detector of the present invention.

FIG. 3 shows an example of a position operating circuit of the radiation detector of the present invention.

FIG. 4 is a position coding map of the radiation detector of the present invention and the existing radiation detector.

FIG. 5 is an energy spectrum of the radiation detector of the present invention.

FIG. 6 shows an identification mechanism of a scintillator array group 11F of the radiation detector of the present invention.

FIG. 7 is an energy spectrum of the scintillator array group 11F of the radiation detector of the present invention.

FIG. 8 shows an identification mechanism of the self-radioactivity of the scintillator array group 11F of the radiation detector of the present invention.

FIG. 9 is an energy spectrum of the self-radioactivity of the scintillator array group 11F of the radiation detector of the present invention.

FIG. 10 is an energy spectrum of a scintillator array group 11R of the radiation detector of the present invention.

FIG. 11 is an energy spectrum of the self-radioactivity of the scintillator array group 11R of the radiation detector of the present invention.

FIG. 12 shows an identification mechanism of the self-radioactivity of a scintillator array of the radiation detector of the present invention.

FIG. 13 is a position coding map of the self-radioactivity of the scintillator array of the radiation detector of the present invention.

FIG. 14 is an energy spectrum of the self-radioactivity of the scintillator array having the scintillators identified to have the shorter attenuation time.

FIG. 15 is an energy spectrum of the self-radioactivity of the scintillator array having the scintillators identified to have the longer attenuation time.

FIG. 16 shows the situation when the γ-ray is incident on the scintillators of the γ-ray detector D.

FIG. 17 shows the situation when the γ-ray is incident on the scintillators of the γ-ray detector MD.

FIG. 18 shows electric signals, i.e., analog signals SF and SR, output from a light receiving element.

FIG. 19 shows a timing of an integral quantity of the digital signals obtained after the analog signals SF and SR are A/D converted.

FIG. 20 shows an identification mechanism of an existing radiation detector.

FIG. 21 shows the identification mechanism of the existing radiation detector.

FIG. 22 shows the identification mechanism of the existing radiation detector.

### DESCRIPTION OF THE SYMBOL LIST

1 scintillator block

1SF scintillators that have a shorter attenuation time of a luminescence pulse

1SR scintillators that have a longer attenuation time of a luminescence pulse

10 radiation detector

11F scintillator array

11R scintillator array

12, 13 light reflective material

15 dark box

20 light guide

31˜34 photomultipliers

35 RI ray source

71˜74 adders

75, 76 position recognizing circuits

80 representing portion on the position coding map

81 position coding map of the scintillator that has a shorter attenuation time

82 position coding map of the scintillator that has a longer attenuation time

83 representing portion on the position coding map 81

84 representing portion on the position coding map 82

110 scintillator array front

111 scintillator array rear

112 two-stage scintillator detector

113 lead collimator jig

114 RI ray source

115 dark box

116 natural radioactive ray

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

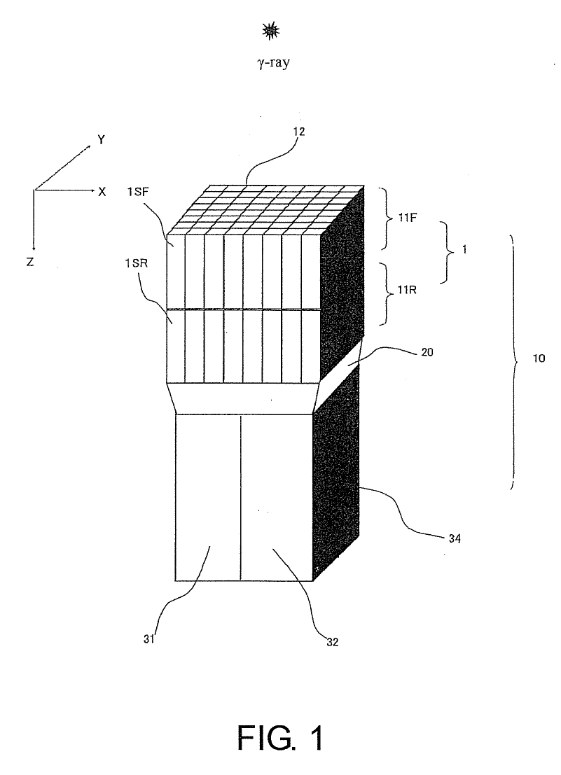

The drawings show the structure of the embodiment of a radiation detector of the present invention, and the detailed illustration is given according to the embodiment. FIG. 1 is an outside view of a radiation detector 10 having scintillator arrays of a two-stage structure of the present invention. The structure of the radiation detector 10 is illustrated with reference to FIG. 1. As shown in FIG. 1, a scintillator block 1 is arranged in partition in the radiation detector 10 in the γ-ray incident depth direction; that is, the radiation detector 10 is a depth of interaction (DOI) detector having three-dimensionally arranged scintillators. The DOI detector of this embodiment has scintillator arrays of a two-stage structure.

The radiation detector 10 of this embodiment is approximately formed by four parts. The first part is a scintillator array 11F having scintillators 1SF that are two-dimensionally and compactly arranged and have a shorter attenuation time of a luminescence pulse. The scintillators 1SF are divided by appropriately sandwiching a light reflective material 12 there-between, and 64 scintillators 1SF are arranged in a manner of eight scintillators in the X direction and eight scintillators in the Y direction. The second part is a scintillator array 11R having scintillators 1SR that are two-dimensionally and compactly arranged and have a longer attenuation time of the luminescence pulse. The scintillators 1SR are divided by appropriately sandwiching a light reflective material 12 there-between, and 64 scintillators 1SR are arranged in a manner of eight scintillators in the X direction and eight scintillators in the Y direction. In one aspect of the invention, the scintillator array 11F and the scintillator array 11R form a scintillator block 1. The third part is a light guide 20, which is optically combined with the scintillator block 1, includes embedded lattice frames combined with a light reflective material 13 (not shown), and is partitioned into a plurality of small areas. The fourth part is four photomultipliers 31, 32, 33, and 34 respectively optically combined with the light guide 20.

Here, the scintillators 1SF having a shorter attenuation time of the luminescence pulse use, for example, inorganic self-radioactive crystals such as Lu₂SiO₅:Ce(LSO), LuYSiO₅:Ce(LYSO), LaBr₃:Ce, LaCl₃:Ce, and LuI:Ce. In another aspect, the scintillators 1SR having a longer attenuation time of the luminescence pulse use inorganic crystals such as Lu_{0.4}Gd_{1.6}SiO₅:Ce(LGSO). Lu has an isotope abundance ratio of Lu-175 97.41% (non-radioactive) and Lu-176 2.59% (radioactive), and is self-radioactive. In another aspect, La has an isotope abundance ratio of La-139 99.911% (non-radioactive) and La-138 0.089% (radioactive), and is self-radioactive. Therefore, the above mentioned scintillators are self-radioactive.

The scintillator block 1 is formed by optically combining the two scintillator arrays 11F and 11R having respectively different attenuation time of the luminescence pulse in the γ-ray incident depth direction (Z direction). The scintillator array 11F has a plurality of scintillators 1SF that is two-dimensionally and compactly arranged and has a shorter attenuation time of the luminescence pulse, and the scintillator array 11R has a plurality of scintillators 1SR that is two-dimensionally and compactly arranged and has a longer attenuation time of the luminescence pulse. Particularly, in the scintillator block 1, for example, LuYSiO₅:Ce(LYSO) is used as the scintillators 1SF having the shorter attenuation time of the luminescence pulse on a γ-ray incident side (front segment), and Lu_{0.4}Gd_{1.6}SiO₅:Ce(LGSO) is used as the scintillators 1SR having the longer attenuation time of the luminescence pulse on the light guide 20 side (back segment).

The two scintillator arrays 11F and 11R are respectively formed by 8×8 (in the X direction and Y direction) chip-shaped scintillators, and the light reflective material 12 or a light transmissive material (not shown), and an optical binding agent (not shown), which enables the light, generated when the γ-ray is incident, to proportionally distribute in the X direction and Y direction, is sandwiched or filled between thereof.

The light guide 20 guides the light generated by the scintillators 11F and 11R of the scintillator block 1 to the photomultipliers 31˜34. The light guide 20 is sandwiched between the scintillator block 1 and the photomultipliers 31˜34 in an optically combining manner by using the optical binding agent.

The light generated by the scintillator arrays 11F and 11R is incident on PMT photoelectric conversion films of four sides and is electronically amplified, and is finally converted to electric signals (analog signals) for output. Therefore, the outputs of the photomultipliers 31˜34 become the output of the radiation detector 10. According to the invention, the light in the scintillator block 1 is guided to the photomultipliers 31˜34 by the optically combined light guide 20, and the position, length, and angle of each light reflective material 13 (not shown) in the light guide 20 are adjusted, such that the output ratio of the photomultiplier 31(33) and the photomultiplier 32(34) arranged in the X direction is changed according to a fixed proportion.

Referring to FIG. 19, a scintillator array identification mechanism is illustrated. FIG. 19 is a graph showing the added value starting from the luminescence pulse to the end of luminescence T2. In addition, in FIG. 19, a curve (A) represents that the light is incident on the scintillators 1SF (scintillator array 11F) having the shorter attenuation time of the luminescence pulse, and a curve (B) represents that the light is incident on the scintillators 1SR (scintillator array 11R) having the longer attenuation time of the luminescence pulse. According to an intermediate added value A_{T1} and a total added value A_{T2}, the intermediate added value A_{T1}/the total added value A_{T2} (the intermediate added value A_{T1} is divided by the total added value A_{T2}) is calculated. A middle value K is set based on a maximum value and a minimum value calculated according to the luminescence pulse emitted from the two scintillator arrays 11F and 11R. Then, according to whether the calculated value is greater or smaller than the middle value K, which one of the scintillator array emits the light is determined. The intermediate added value A_{T1} is a value obtained by adding the digital signals until the middle of a period starting from the luminescence pulse being emitted from the scintillator arrays 11F and 11R to the end of luminescence T2, in other words, until an intermediate moment T1. The total added value A_{T2} is a value obtained at an intermediate moment T₁ by adding the digital signals during a period starting from the luminescence pulse being emitted from the scintillator arrays 11F and 11R to the end of luminescence T2. In addition, A_{T1}/A_{T2} is calculated, and if the calculated result is greater than the middle value K, it is identified as the scintillator array 11F having the shorter attenuation time; on the contrary, if the calculated result is smaller than the middle value K, it is identified as the scintillator array 11R having the longer attenuation time.

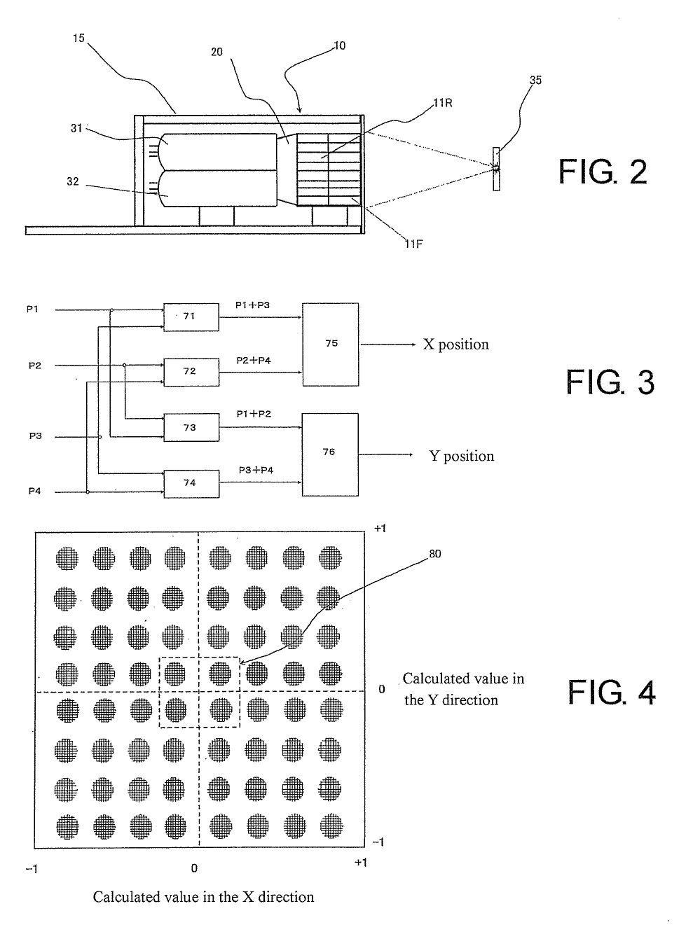

In the present invention, the parameters T1, T2, and K required by the scintillator array identification mechanism are determined in the following manner. As shown in FIG. 2, the RI ray source 35 irradiates from the front the γ-ray on the radiation detector 10 having scintillator arrays of a two-stage structure and disposed in the dark box 15, and a position coding map and an energy spectrum are obtained. That is, if the output of the photomultiplier 31 is set to P1, the output of the photomultiplier 32 is set to P2, the output of the photomultiplier 33 is set to P3, and the output of the photomultiplier 34 is set to P4, a calculated value {(P1+P3)-(P2+P4)}/(P1+P2+P3+P4) representing a position in the X direction is obtained, and a calculated value {(P1+P2)-(P3+P4)}/(P1+P2+P3+P4) representing a position in the Y direction is also obtained.

FIG. 3 is a block diagram of a structure of a position operating circuit of the radiation detector 10. The position operating circuit is formed by adders 71, 72, 73, and 74, and position recognizing circuits 75 and 76. As shown in FIG. 3, in order to detect the incident position of the γ-ray in the X direction, the output P1 of the photomultiplier 31 and the output P3 of the photomultiplier 33 are input to the adder 71, and the output P2 of the photomultiplier 32 and the output P4 of the photomultiplier 34 are input to the adder 72. Each added output (P1+P3) and (P2+P4) of the two adders 71 and 72 are input to the position recognizing circuit 75. According to the two added outputs, the incident position of the γ-ray in the X direction is determined. Similarly, in order to detect the incident position of the γ-ray in the Y direction, each added output (P1+P2) and (P3+P4) are input to the position recognizing circuit 76. According to the two added outputs, the incident position of the γ-ray in the Y direction is determined. According to the positions of the γ-ray incident on the scintillators, the calculated results obtained above are represented by the position coding map showing the position recognition information, as shown in FIG. 4.

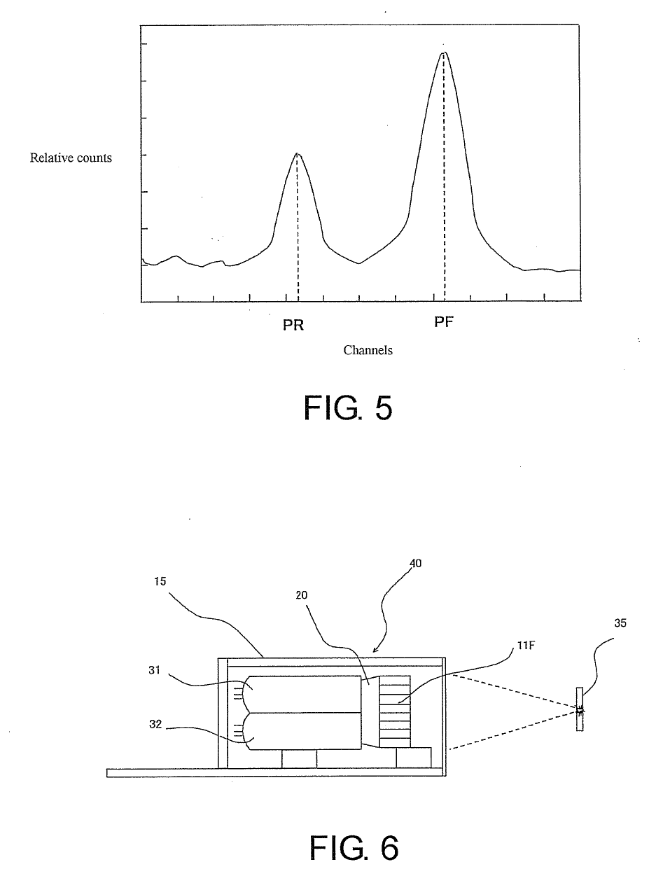

In another aspect, the calculated value (P1+P2+P3+P4) represents the energy relative to the event, and is calculated as the energy spectrum. FIG. 5 is an energy spectrum relative to a representing portion 80 on the position coding map. According to this aspect of the invention, the luminescence outputs of the two scintillators are different, and two energy peak values PF and PR occur. In this embodiment, PF is equivalent to LYSO and PR is equivalent to LGSO.

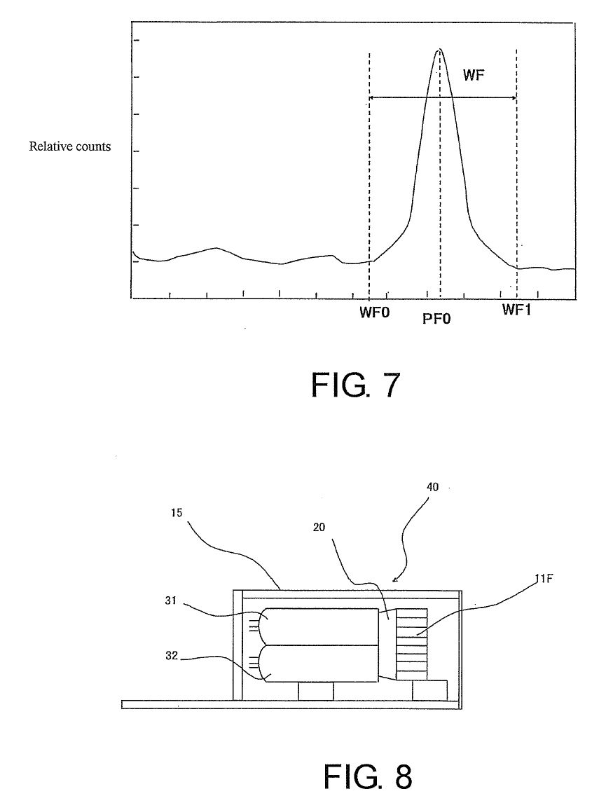

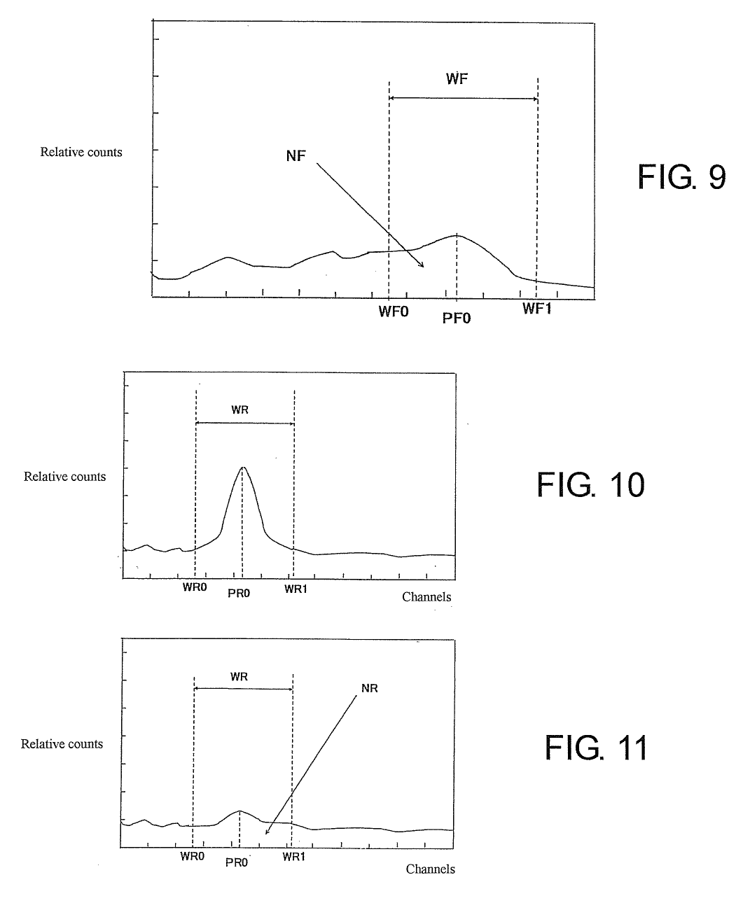

Then, as shown in FIG. 6, the RI ray source 35 irradiates the γ-ray from the front on the radiation detector 40 formed by only the scintillator array 11F, and a position coding map and an energy spectrum are obtained. Similarly, FIG. 7 is an energy spectrum relative to the representing portion 80 on the position coding map. As shown in FIG. 7, an energy peak value PF0 resulting from the scintillator array 11F, i.e. LYSO, occurs, but the gain of the photomultipliers is adjusted to a value at which PF0 is the same as PF. In this state, an energy window WF decided by channels WF0˜WF1 is determined, such that the energy window WF includes an energy peak value count. Then, the RI ray source 35 is removed, as shown in FIG. 8, and for the radiation detector 40 only formed by the scintillator array 11F, the position coding map and the energy spectrum resulted from the self-luminescence based on the self-radioactivity are measured. FIG. 9 is an energy spectrum relative to the representing portion 80 on the position coding map. In FIG. 9, the energy spectrum resulted from the self-luminescence based on the self-radioactivity of the scintillator array 11F, i.e. LYSO is presented, and the count NF in the energy window WF determined by the channels WF0˜WF1 is calculated.

Further, the RI ray source 35 irradiates the γ-ray from the front on the radiation detector 40 formed by only the scintillator array 11R, and a position coding map and an energy spectrum are measured. Similarly, FIG. 10 is an energy spectrum relative to the representing portion 80 on the position coding map. As shown in FIG. 10, an energy peak value PR0 resulting from the scintillator array 11R, i.e. LGSO is identified, but the gain of the photomultipliers is adjusted to a value at which PR0 is the same as PR. In this state, an energy window WR decided by channels WR0˜WR1 is determined, such that the energy window WR includes an energy peak value count. Then, the RI ray source 35 is removed, and for the radiation detector 40 formed by only the scintillator array 11R, the position coding map and the energy spectrum caused by self-luminescence based on the self-radioactivity are measured. FIG. 11 is an energy spectrum relative to the representing portion 80 on the position coding map. In FIG. 11, the energy spectrum resulted from self-luminescence based on the self-radioactivity of the scintillator array 11R, i.e. LGSO, is presented, and the count NR in the energy window WR determined by the channels WR0˜WR1 is calculated.

NF/NR is defined by NF and NR.

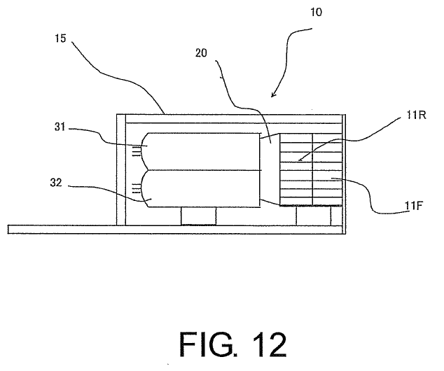

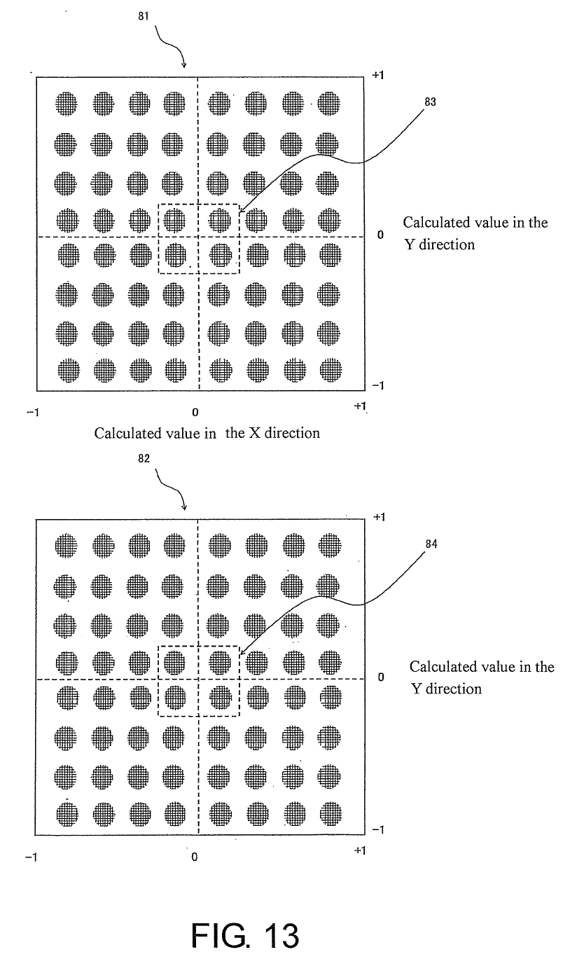

Then, the parameters T1, T2, and K required by the scintillator array identification mechanism are temporarily determined, as shown in FIG. 12. For the radiation detector 10 having the scintillator arrays of a two-stage structure and disposed in the dark box 15, a position coding map and an energy spectrum resulted from self-luminescence based on the self-radioactivity are measured. The parameters T1, T2, and K are pre-determined, so as shown in FIG. 13 the position coding map represents a position coding map 81 of the scintillator array that is identified to have the shorter attenuation time and a position coding map 82 of the scintillator array that is identified to have the longer attenuation time.

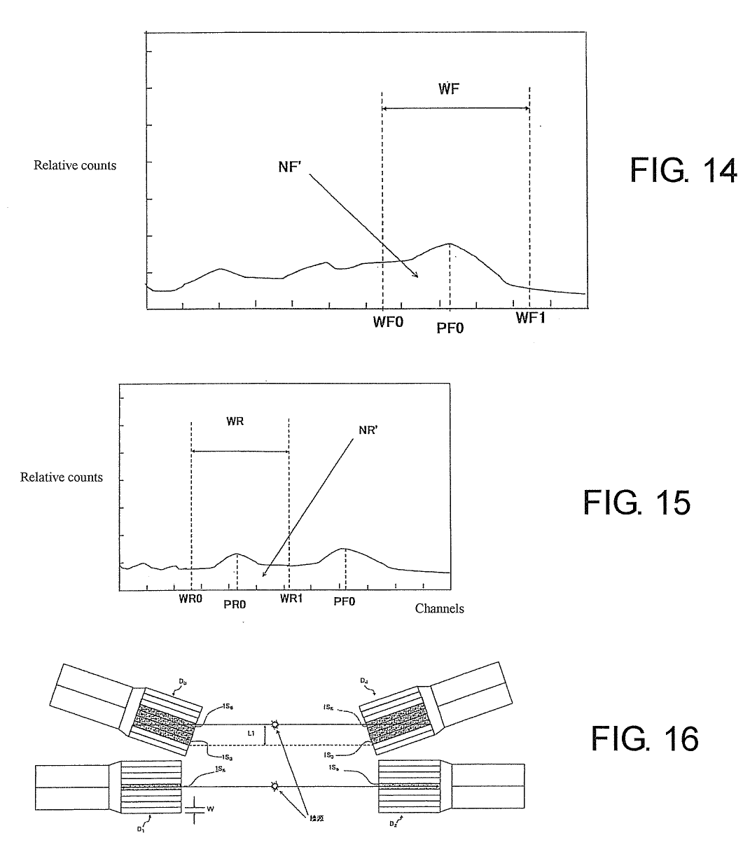

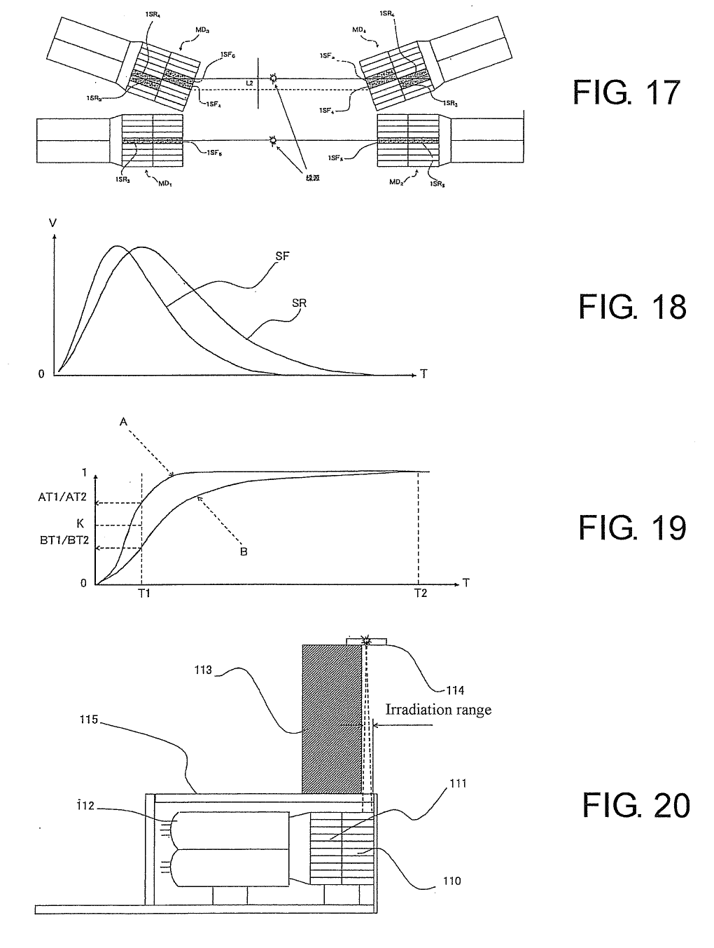

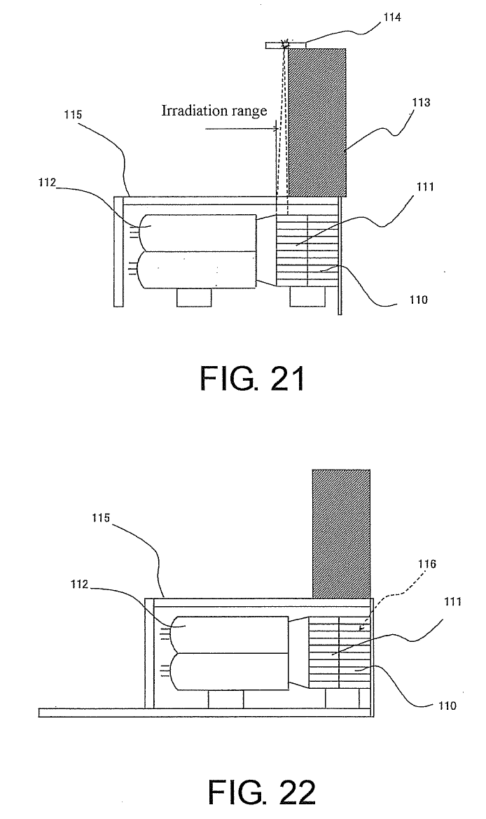

Here, FIG. 14 is an energy spectrum relative to a representing portion 83 on the position coding map 81, and FIG. 15 is an energy spectrum relative to a representing portion 84 on the position coding map 82. If the parameters T1, T2, and K are optimal, the energy spectrum of FIG. 14 is supposedly the energy spectrum resulted from self-luminescence based on the self-radioactivity of the scintillator array 11F, i.e. LYSO. In another aspect, if the parameters T1, T2, and K are optimal, the energy spectrum of FIG. 15 is supposedly the energy spectrum resulted from the self-luminescence based on the self-radioactivity of the scintillator array 11R, i.e. LGSO. However, in the energy spectrum of FIG. 15, the energy spectrum resulted from self-luminescence based on the self-radioactivity of the LGSO occurs near the channel PF0. Hence, it is estimated that the parameters T1, T2, and K are not optimal.

A count NF′ in the energy window WF determined by the channels WF0˜WF1 in the energy spectrum of FIG. 14 is calculated, and a count NR′ in the energy window WR determined by the channels WR0˜WR1 in the energy spectrum of FIG. 15 is calculated.

Further, NF′/NR′ is defined by NF′ and NR′. Although it is ultimately required to optimize the parameters T1, T2, and K required by the scintillator array identification mechanism, as the indexes thereof, the parameters may be decided in the manner of satisfying NF/NR=NF′/NR′.

In the case of such an identification mechanism, the radioactivity of the self-radioactive scintillators is fixed; therefore, once the data of NF and NR are obtained, the other lots of scintillators may also use the same data of NF and NR. That is, once the data of NF and NR are obtained, a lead collimator jig and an RI ray source used only for irradiating the γ-ray on any one scintillator array as in the previous example are not required. Hence, the operation is not simpler, and the management of the RI ray source is unnecessary. Further, after a detector is practically loaded on the PET device, the large lead collimator jig and RI ray source matching the PET device are not required, and the operation is simple.

The present invention is not limited to the embodiments, and may also be implemented in the following variations.

In the embodiments above, the PET device is used as an example for illustration. However, the present invention may be applied in other nuclear medical diagnostic devices besides the PET device, as long as the devices may perform a simultaneous counting for the radioactive rays emitted from the detected body on which the radioactive agent is applied.

The embodiments of the present invention may be applied to a device formed by combining the nuclear medical diagnostic device and an X-ray CT device, such as a PET-CT having the PET device and the X-ray CT device.

In the embodiments above, the scintillator block 1 is formed by combining the two (layers of) scintillator arrays 11F and 11R, but may also be formed by combining a plurality of (layers of) scintillator arrays in addition to the two (layers of) scintillator arrays. Further, the number of the scintillators in each of the scintillator arrays 11F and 11R is 8×8, but may also be a multiple besides 8×8.

In the embodiments, the light receiving element is the photomultipliers 31˜34, but other light receiving elements may also be used, for example, photodiodes or avalanche photodiodes etc.

### INDUSTRIAL AVAILABILITY

As described above, the present invention is suitable for medical and industrial radiation imaging devices.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A nuclear medical diagnostic device, comprising:
a scintillator block, formed by optically combining a plurality of self-radioactive scintillator arrays, wherein the plurality of the scintillator arrays comprises several scintillators that are two-dimensionally and compactly arranged, wherein attenuation times of luminescence pulses of the several scintillators in a γ-ray incident depth direction are different;
a light receiving element, for converting the luminescence pulses emitted from the scintillator block into electric signals;
an A/D converter, for converting the electric signals output from the light receiving element into digital signals; and
an identification mechanism, for identifying the plurality of scintillator arrays with the different attenuation times of the luminescence pulses in the γ-ray incident depth direction,
wherein parameters required by the scintillator array identification mechanism are determined with reference to a signal count of the self-radioactivity of the self-radioactive scintillator arrays.

2. (canceled)

3. The nuclear medical diagnostic device according to claim 1, wherein the scintillator array identification mechanism comprises:
an adding mechanism, for adding sequentially the digital signals converted by the A/D converter;
an identification value calculating mechanism, for calculating an identification value representing a value obtained by dividing an intermediate added value by a total added value, wherein the intermediate added value is a value obtained by adding the digital signals until a middle time period starting from the luminescence pulses emitted from the scintillator block to an end of an luminescence, which is an intermediate moment in the adding mechanism, and the total added value is a value obtained by adding the digital signals during a period starting from the luminescence pulse emitted from the scintillator block to the end of the luminescence in the adding mechanism;
a middle value calculating mechanism, for calculating a middle value according to a maximum value and a minimum value in the identification value calculated by the identification value calculating mechanism; and
a determination mechanism, for determining whether an identification value calculated by the identification value calculating mechanism is greater or smaller than the middle value calculated by the middle value calculating mechanism,
wherein a determination method comprises determining parameters of the middle time period and the middle value with reference to a signal count of a self-radioactivity of the self-radioactive scintillator arrays.

4. The nuclear medical diagnostic device according to claim 1, comprising a light guide that optically combines the scintillator block and the light receiving element.
